(19) United States
(12) Patent Application Publication
(10) Pub. No.: US 2024/0374441 A1
(43) Pub. Date: Nov. 14, 2024

(54) ABSORBENT ARTICLE

(71) Applicant: The Procter & Gamble Company, Cincinnati, OH (US)

(72) Inventors: Zhengtao LI, Beijing (CN); Gueltekin ERDEM, Cincinnati, OH (US); Zheng BAO, Beijing (CN); Yibo FAN, Beijing (CN)

(21) Appl. No.: 18/655,376

(22) Filed: May 6, 2024

(30) Foreign Application Priority Data
May 12, 2023 (WO) PCT/CN2023/093827
Apr. 12, 2024 (WO) PCT/CN2024/087454

(51) Int. Cl. A61F 13/537; A61F 13/15; A61F 13/53

(52) U.S. Cl. CPC A61F 13/5376; A61F 13/53708; A61F 2013/15422; A61F 2013/15487; A61F 2013/15552; A61F 2013/530233; A61F 2013/530379

(57) ABSTRACT

The present invention relates to a thin, flexible absorbent article which comprises a topsheet, a backsheet, an absorbent core disposed between the topsheet and the backsheet, and an acquisition-distribution system (“ADS”) disposed between the topsheet and the absorbent core. The ADS comprises a first layer comprising a nonwoven. The first layer comprises a first stratum facing towards the topsheet and comprising first absorbing fibers, a second stratum facing towards the backsheet and comprising second absorbing fibers, and a plurality of apertures. A percentage of the second absorbing fibers in the second stratum is at least about 15% higher than a percentage of the first absorbing fibers in the first-stratum as measured according to the Fiber Number Test.

### CROSS REFERENCE TO RELATED APPLICATIONS

This application claims priority under 35 U.S.C. § 119 to Chinese Patent Application Serial No. PCT/CN2024/087454, filed on Apr. 12, 2024 and PCT/CN2023/093827, filed on May 12, 2023, the entire disclosures of both of which are hereby incorporated by reference.

### FIELD

The invention relates to absorbent articles which are thin, flexible, and comfortable and a high wet air permeability nevertheless have a high capacity to absorb fluids and have improved in run-off.

### BACKGROUND

Disposable absorbent articles such as diapers and adult incontinence products are well known in the art. Such disposable articles are designed to absorb and contain body exudates, in particular large quantity of urine. These absorbent articles may comprise several layers providing different functions, for example, a topsheet, a backsheet, an absorbent core disposed between the topsheet and the backsheet, and an acquisition-distribution system disposed between the topsheet and the absorbent core among other layers.

One function of an absorbent core is to absorb and retain the bodily exudates for a prolonged amount of time, for example, overnight for a diaper, minimize re-wet to keep the wearer dry, and avoid soiling of clothes or bed sheets. Some currently marketed absorbent articles comprise absorbent cores comprising an absorbent material which is a blend of comminuted wood pulp (i.e., airfelt) with superabsorbent polymers (SAP) in particulate form, also called absorbent gelling materials. Absorbent articles having a core consisting essentially of SAP as the absorbent material (so called “airfelt-free” cores) have also been proposed.

One function of an acquisition-distribution system (“ADS”) is to quickly acquire liquids or other bodily exudates and distribute them to the absorbent core in an efficient manner. The ADS may comprise one or more layers which may form a unitary structure, or may comprise discrete layers. Some currently marketed absorbent articles comprise an ADS comprising a nonwoven layer and/or a cellulose-containing layer.

There are two specific challenges in delivering thin, flexible, comfort yet highly absorbent articles; the first is having sufficient fluid handling properties such as a high acquisition speed and a mitigated rewet and the second is making the absorbent article thin, flexible and comfortable.

Traditionally, highly absorbent products such as incontinence or diaper products are relatively thick in order to absorb high amounts of discharge delivered quickly. More recently, absorbent articles comprising an airfelt-free core were developed. These absorbent articles have a relatively low caliper and still remain to have a high absorbency but are invariably stiffer and harder. Some thinner products having improved flexibility are hardly able to provide a desired fluid absorbency and a low rewet. These thinner products having a relatively low acquisition speed tend to have a high cuff force to prevent fluid leakage caused by the low acquisition speed which may cause skin markings and irritation when worn by a wearer.

Based on the foregoing, there is a need for absorbent articles which are thin, flexible and are able to sustain absorption speed properties.

Based on the foregoing, there is a need for absorbent articles which are thin, flexible and comfortable, and are able to reduce rewet.

Based on the foregoing, there is a need for absorbent articles which are thin, flexible and are able to allow to stay at a low cuff force range.

### SUMMARY

The present invention relates to an absorbent article comprising a topsheet, a backsheet, an absorbent core disposed between the topsheet and the backsheet, and an acquisition-distribution system (“ADS”) disposed between the topsheet and the absorbent core, the ADS comprising a first layer comprising nonwoven, wherein the first layer comprises a first stratum facing towards the topsheet and comprising first absorbing fibers, a second stratum facing towards the backsheet and comprising second absorbing fibers, and a plurality of apertures; and wherein the percentage of the second absorbing fibers in the second stratum is at least about 15% higher than, or at least about 17% higher than, or at least 19% higher than the percentage of the first absorbing fibers in the first stratum as measured according to the Fiber Number Test.

The present invention also relates to a nonwoven comprising a first layer comprising a first stratum and a second stratum, the first stratum comprising first absorbing fibers and synthetic fibers, a second stratum comprising second absorbing fibers and synthetic fibers, and a plurality of apertures; and, wherein the percentage of the second absorbing fibers in the second stratum is at least about 15% higher than, or at least about 17% higher than, or at least 19% higher than the percentage of the first absorbing fibers in the first stratum as measured according to the Fiber Number Test.

The article is illustrated in the Figures as a taped diaper. For ease of discussion, the absorbent article and the acquisition-distribution system will be discussed with reference to the numerals referred to in these Figures. The Figures and detailed description should however not be considered limiting the scope of the claims, unless explicitly indicated otherwise, and the invention disclosed herein is also used in a wide variety of absorbent article forms.

### BRIEF DESCRIPTION OF THE DRAWINGS

In the drawings, like numerals or other designations designate like features throughout the views.

FIG. 1 is a schematic plan view of an exemplary absorbent article according to the present invention.

FIG. 2 is a lateral cross-section along 2-2 of the absorbent article of FIG. 1.

FIG. 3 is a schematic cross-section view of an exemplary acquisition-distribution system for an absorbent article according to the present invention.

FIG. 4 is a schematic cross-section view of an exemplary acquisition-distribution system for an absorbent article according to the present invention.

FIG. 5 is a schematic representation of a process which can be utilized to construct nonwoven according to the present invention.

FIG. 6 is a microscopic image of a first surface of a first layer of the ADS disclosed herein.

FIG. 7A is an SEM image of a first surface of a first layer of the ADS disclosed herein.

FIG. 7B is an enlarged SEM image of a first surface of a first layer of the ADS disclosed herein.

FIG. 8A is an SEM image of a second surface of a first layer of the ADS disclosed herein.

FIG. 8B is an enlarged SEM image of a second surface a first layer of the ADS disclosed herein.

FIG. 9 is a microscopic image of a first surface of a comparison nonwoven.

FIG. 10A is an SEM image of a first surface of a comparison nonwoven.

FIG. 10B is an enlarged SEM image of a first surface of a comparison nonwoven.

FIG. 11A is an SEM image of a second surface of a comparison nonwoven.

FIG. 11B is an enlarged SEM image of a second surface of a comparison nonwoven.

FIG. 12 illustrates an apparatus used in the Modified Fluid Acquisition Test.

FIG. 13A is a side view of the curved component used in the Modified Fluid Acquisition Test.

FIG. 13B is an end view of the curved component of FIG. 13A.

FIG. 13C is a bottom view of the curved component of FIG. 13A.

FIG. 13D is a bottom perspective view of the curved component of FIG. 13A.

FIG. 13E is a top perspective view of the curved component of FIG. 13A.

FIG. 14A illustrates a top plate assembly used in the Modified Fluid Acquisition Test.

FIG. 14B illustrates equipment used in the Modified Fluid Acquisition Test.

### DETAILED DESCRIPTION

Various non-limiting forms of the present disclosure will now be described to provide an overall understanding of the principles of the structure, function, manufacture, and use of an absorbent article according to the present invention. One or more examples of these non-limiting embodiments are illustrated in the accompanying drawings. The features illustrated or described in connection with one non-limiting form may be combined with the features of other non-limiting forms. Such modifications and variations are intended to be included within the scope of the present disclosure.

### Definitions of Terms

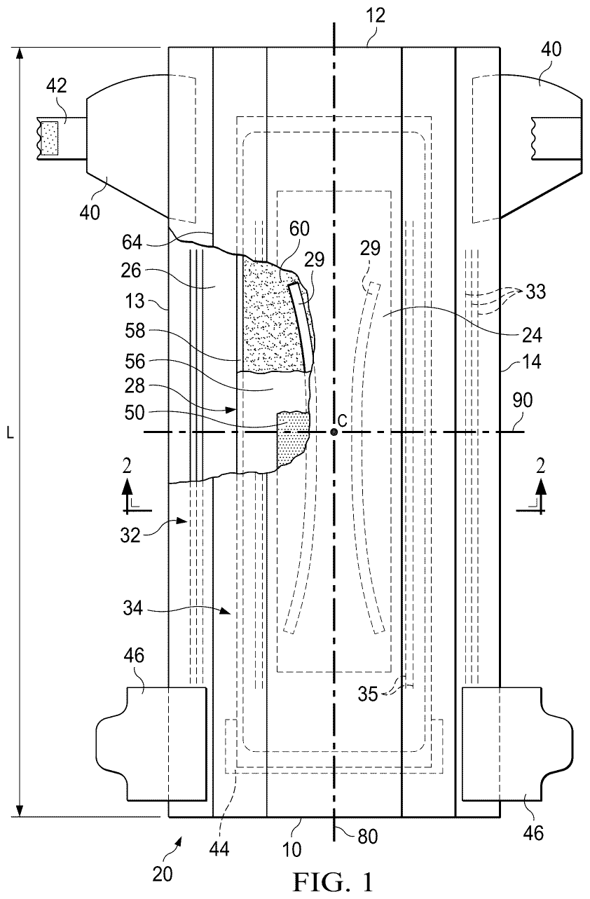

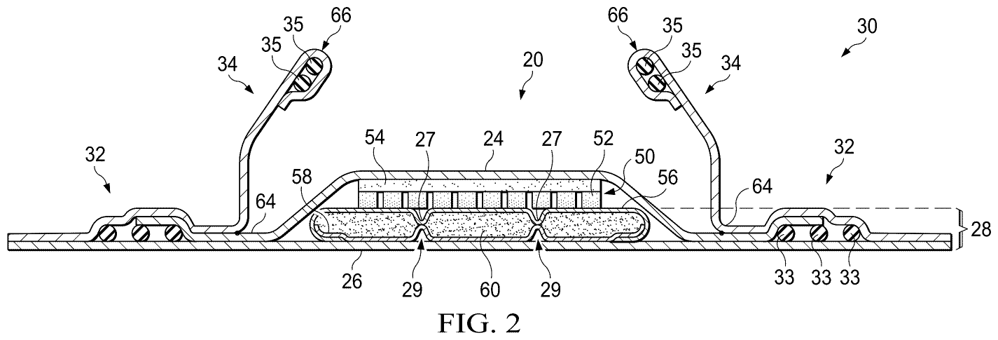

The term “absorbent article” as used herein refers to disposable products such as taped diapers, diapers having a closed waist opening (pants), adult incontinent products, hygiene sanitary napkins and the like, which are placed against or in proximity to the body of the wearer to absorb and contain bodily exudates such as urine, feces or menses discharged from the body. Typical absorbent articles comprise a topsheet, a backsheet, an absorbent core, an acquisition layer and other components. The liquid permeable topsheet forms at least a portion of the wearer-facing side of the article, and the liquid impermeable backsheet forms at least a portion, and typically the whole, of the garment-side of the article. The articles may be provided with fastening elements, such as tapes (taped diapers) or may be provided already pre-formed with a waist opening and a pair of leg openings as in an underwear (pant diapers). The absorbent articles may be for use with babies, infants, women or incontinent adults. Typical features of absorbent articles are further discussed further below, and in relation with the illustrated taped diaper in FIGS. 1 and 2, which is of course for illustration purpose only and not limiting the scope of the inventions, unless specifically indicated otherwise.

The term “air-through bonding” means passing a material such as fibrous web to be bonded through a stream of heated gas, such as air, in which the temperature of the heated gas is above the softening or melting temperature of at least one polymer component of the material being bonding air-through bonding may involve passing a material through a heated oven.

The term “aperture”, as used herein, refers to a hole. The apertures can be holes cleanly through the web so that the material surrounding the aperture lies in the same plane as the web prior to the formation of the aperture (a “two dimensional” aperture). The apertures can also be holes which is protruded out of the plane of the web (a “three dimensional” aperture).

The term “carded web” or “carded nonwoven” means a web or nonwoven comprising staple fibers that are predominantly aligned and oriented in the machine direction using a carding process.

The term “composite” means a structure comprising two or more layers. The two or more layers of a composite may be joined together such that a substantial portion of their common X-Y plane interface.

The words “typically”, “normally”, “advantageously” and the likes also qualify features which are not intended to limit the scope of the claims unless specifically indicated to do so.

The term “integrated” as used herein is used to describe fibers of a nonwoven material which have been intertwined, entangled, and/or pushed/pulled in a positive and/or negative Z-direction (direction of the thickness of the nonwoven material). Some exemplary processes for integrating fibers of a stratum include spunlacing and needlepunching. Spunlacing uses a plurality of high pressure water jets to entangle fibers. Needlepunching involves the use of needles to push and/or pull fibers to entangle them with other fibers in the nonwoven.

“Machine direction” or “MD” is the direction parallel to the direction of travel of the web in a manufacturing process. The machine direction is typically the longitudinal direction of a component, such as an ear of an absorbent article. The “cross machine direction” or “CD” is the direction substantially perpendicular to the MD and in the plane generally defined by the web.

“Natural fibers” refers to elongated substances produced by plants and animals and includes animal-based fibers and plant-based fibers, as those categories are described herein.

Natural fibers, as that term is used herein, include fibers harvested without any post-harvest treatment step as well as those having a post-treatment step, such as, for example, washing, scouring, bleaching.

The term “nonwoven” as used herein refers to a manufactured material, web, sheet or batt of directionally or randomly oriented fibers, bonded by friction, and/or cohesion and/or adhesion, excluding paper and products which are woven, knitted, tufted, stitch-bonded, incorporating binding yarns or filaments, or felted by wet milling, whether or not additionally needled. The fibers may be of natural or man-made origin. The fibers may be staple or continuous filaments or be formed in situ. The porous, fibrous structure of a nonwoven may be configured to be liquid permeable or impermeable, as desired.

The term “staple fiber” means a fiber having a finite length. In general, staple fibers may have a length from about 2 to 200 mm.

### Absorbent Articles

Absorbent articles will now be generally discussed and further illustrated in the form of a baby diaper 20 as exemplarily represented in FIGS. 1 and 2. FIG. 1 is a plan view of the exemplary diaper 20 in a flattened-out configuration with the taped ends opened and the garment-facing side turned up. An article that is presented to the user closed such as a training pant may also be represented flattened out by cutting it along its side waists. This diaper is shown for illustration purpose only as the present invention is applicable to a wide variety of diapers or other absorbent articles. The absorbent article 20 notionally comprises a front edge 10, a back edge 12 and longitudinally-extending first and second longitudinal side edges 13, 14. The front edge 10 forms the edge of the front waist and the back edge 12 of the back waist, which together when worn by the wearer form the opening for the waist of the wearer. The longitudinal side edges 13, 14 can each form one of the leg openings. The absorbent article 20 notionally comprises a longitudinally centerline 80 dividing the article in a left side and a right side, and a perpendicular transversal centerline 90 disposed at half the length of the article as measured on the longitudinal centerline 80, with both centerlines crossing at the center point C. The taped back ends 42 attached on the front of the diaper to such as a landing zone 44.

Referring to FIGS. 1 and 2, the absorbent article 20 includes, from top to bottom, a topsheet 24, an acquisition-distribution system 50, an absorbent core 28 and a backsheet 26. A suitable topsheet may be manufactured from a wide range of materials, such as porous foams, reticulated foams, apertured plastic films, woven materials, nonwoven materials, woven or nonwoven materials of natural fibers (e.g., wood or cotton fibers) or regenerated fibers such as viscose fibers, synthetic fibers or filaments (e.g., polyester or polypropylene or bicomponent PE/PP fibers or mixtures thereof), or a combination of natural and synthetic fibers.

The absorbent article 20 further comprises a leg gasketing system 30 comprising an inner and outer cuffs 34, 32, which are preferably elasticized with elastic strands 35, 33 respectively. Elasticized back ears 40 having a tape end 42 can be attached to a landing zone 44 at the front of the article. Front ears 46 are typically present in such taped diapers to improve containment and attachment. An outer cover, not indicated in the drawings, may cover at least a portion of, or all of, the backsheet 26 to form a soft garment-facing surface of the absorbent article. The outer cover may be formed of one or multi-layered nonwoven. The nonwoven can comprise a combination of natural fibers and synthetic fibers that are not natural fibers. For example, the nonwoven can comprise both polypropylene fibers and cotton fibers; see, for example, U.S. Patent Application Publication No. U.S. 2017/0203542. The outer cover may be joined to at least a portion of the backsheet 26 through mechanical bonding, adhesive bonding, or other suitable methods of attachment. The backsheet 26 and/or the outer cover may comprise apertures which can promote a breathability conception.

The absorbent article may further comprise a nonwoven layer disposed between the absorbent core and the backsheet. The nonwoven layer may improve undesirable grainy feel and provide a cushiony softness to the absorbent article.

The absorbent article may also comprise other typical components, which are not represented, such as a back-elastic waist feature, a front elastic waist feature, transverse barrier cuff(s), a lotion application, etc.

### Absorbent Core

As used herein, the term “absorbent core” refers to a component used or intended to be used in an absorbent article and which comprises an absorbent material and optionally a core wrap. As used herein, the term “absorbent core” does not include the topsheet, the backsheet and any acquisition-distribution layer or multilayer system, which is not integral part of the absorbent core. The absorbent core is typically the component of an absorbent article that has the most absorbent capacity of all the components of the absorbent article. The terms “absorbent core” and “core” are herein used interchangeably.

Referring to FIGS. 1 and 2, the absorbent core 28 can absorb and contain liquid received by the absorbent article and comprise an absorbent material 60, which may be absorbing fibers, a blend of superabsorbent polymers and absorbing fibers, pure superabsorbent polymers, and/or a high internal phase emulsion foam.

The absorbent core 28 may comprise at least one channel 29 free of an absorbent material, or may comprises multiple absorbent material free channels 29 as shown in FIGS. 1 and 2. In an embodiment, the top side 56 of the core wrap may be bonded to the bottom side 58 of the core wrap through the channel 29. The core wrap bonds 27 may at least persist as the absorbent core 28 swells upon liquid absorption and creates three-dimensional channels at the wearer-facing surface of the article. Further details regarding channels are disclosed in WO2015/134359A. Of course, this is entirely optional, the absorbent core may also not have bonded channels, or even unbonded channels.

The absorbent material defines an absorbent material area, which may be rectangular as show in in FIG. 1, but it is also common to have a shaped area which is tapered in the area around the transversal centerline 90. The patent literature is replete with example of such and other components suitable for use in the diapers of the invention, see for example already referred to WO2017/156200A and WO2019/076288A and these will not be discussed in extension herein.

The absorbent material comprises a liquid-absorbent material commonly used in disposable absorbent articles such as comminuted wood pulp, which is generally referred to as airfelt or fluff. Examples of other suitable liquid-absorbent materials include creped cellulose wadding; melt blown polymers, including co-form; chemically stiffened, modified or cross-linked cellulosic fibers; tissue, including tissue wraps and tissue laminates, absorbent foams, absorbent sponges, superabsorbent polymers (herein abbreviated as “SAP”), absorbent gelling materials, or any other known absorbent material or combinations of materials.

The absorbent material in the absorbent core can be any type. For absorbent cores comprising a relatively high proportion of SAP, the SAP content may represent in particular at least 80%, 85%, 90%, 95% and up to 100%, of superabsorbent polymer by weight of the absorbent material. The absorbent material may in particular comprise no or only small amount of absorbing fibers, such as less than 20%, in particular less than 10%, 5% or even 0% of absorbing fibers by weight of the absorbent material. The absorbent core may comprise an absorbent material comprising at least 80%, at least 90%, at least 95%, or at least 99% by weight of the absorbent core. The term “superabsorbent polymer” refers herein to absorbent material, which may be cross-linked polymer, and that can typically absorb at least 10 times their weight of an aqueous 0.9% saline solution as measured using the Centrifuge Retention Capacity (CRC) test (EDANA method WSP 241.2-05E). The SAP may in particular have a CRC value of more than 20 g/g, or more than 24 g/g, or of from 20 to 50 g/g, or from 20 to 40 g/g, or from 24 to 30 g/g. The SAP may be typically in particulate forms (superabsorbent polymer particles), but it is not excluded that other forms of SAP may be used such as a superabsorbent polymer foam for example.

### Acquisition-Distribution System

Referring to FIGS. 1 and 2, the absorbent article 20 of the present disclosure comprises an acquisition-distribution system (“ADS”) 50. One function of ADS 50 is to quickly acquire the body fluid such as urine and distribute them to the absorbent core 28 in an efficient manner. Further, having an ADS of the present invention, the absorbent article may have a low run-off of liquid. Therefore, the risk of leakage of the absorbent article is reduced when the ADS disclosed herein invention is used in an absorbent article.

The ADS 50 may comprise a single layer. It may have two or more layers, which may form a unitary structure or may remain as discrete layers which may be attached to each other by, for example, thermal bonding, adhesive bonding or a combination thereof. A unitary structure herein intends to mean that although it may be formed by several sub-layers that have distinct properties and/or compositions from one another, they are somehow intermixed at the boundary region so that, instead of a definite boundary between sub-layers, it would be possible to identify a region where the different sub-layers transition one into the other. Such a unitary structure is typically built by forming the various sub-layers one on top of the other in a continuous manner, for example using air laid or wet laid deposition. Typically, there is no adhesive used between the sub-layers of the unitary material. However, in some cases, adhesives and/or binders can be present although typically in a lower amount that in multilayer materials formed by separate layers.

The ADS according to the present invention comprises a first layer comprising absorbing fibers and synthetic fibers. Without being bound by theory, the absorbing fibers provide liquid storage capability and provide a springy open structure that enables quick recovery of the ADS to enable readiness for multiple assaults. The synthetic fibers may enhance structural integrity of the ADS while also providing for a more open structure.

The first layer comprises at least two stratums integrated without adhesives.

The ADS may further comprise a second layer comprising thermoplastic fibers in such a way the second layer is positioned between the topsheet and the first layer of the ADS. The ADS for the present invention, when comprising the first layer and the second layer, may further comprise one or more additional layers deposited on the bottom surface of the first layer, or on the top surface of the second layer, or between the first layer and the second layer.

A wide variety of configurations for an ADS may be achieved. However, it is important that the ADS of the present disclosure has adequate openness to allow for quick acquisition of fluid, yet also be able to lock away liquid insults to reduce the likelihood of rewet.

The ADS of the present disclosure can have a basis weight of up to 150 grams per square meter (gsm), of up to 100 gsm; or between about 20 gsm to about 120 gsm; or between about 30 gsm to about 100 gsm, or between from about 45 gsm to about 80 gsm, and or between about 50 gsm to about 65 gsm, including any values within these ranges and any ranges created thereby. Some absorbent articles may not require as much basis weight as recited above. For example, liners which generally do not have the same level of absorbent capacity as menstrual pads may be able to have a reduced basis weight over that which was recited above. For example, the ADS may have a basis weight of between 20 gsm to 70 gsm or between 35 gsm to about 65 gsm, or from about 30 gsm to about 55 gsm, specifically including all values within these ranges and any ranges created thereby.

ADS may be spunlaced that is produced via conventional spunlace processes widely known in the industry.

In one embodiment, the ADS may be bonded to adjacent components in the absorbent article using adhesives. In another embodiment, the ADS may not require adhesives or latex binders for stability.

### First Layer

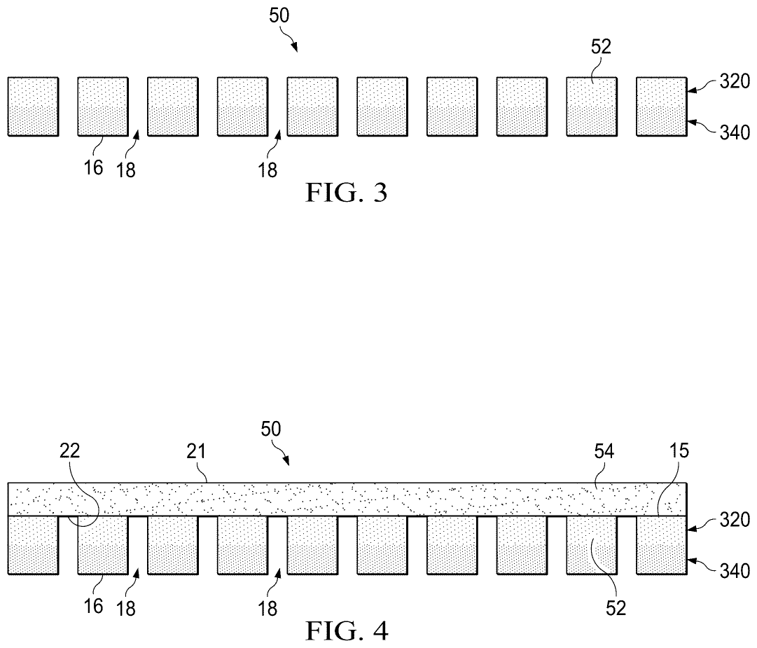

The first layer comprises absorbing fibers and synthetic fibers such as thermoplastic fibers. FIG. 3 is a schematic representation of an exemplary first layer 52. The first layer 52 comprises a first stratum 320 and a second stratum 340, and has a first surface (top surface) 15, an opposite second surface (bottom surface) 16 and a plurality of apertures 18. The first stratum 320 forms the first surface 15 closest to the wearer-facing surface in an absorbent article, and the second stratum 340 forms the opposite second surface 16.

The absorbing fibers are not higher than about 95%, or from about 50% to about 85%, or from about 60% to about 80%, or from about 15% to about 60%, by weight of the first layer. When the amount of absorbing fibers is higher than 95%, it may render the first layer to collapse when the ADS gets wet, therefore an absorbent speed may be deteriorated. When the amount of absorbing fibers is too low, for example lower than 15%, ADS may not have enough void volume for temporarily storing the fluid initially which may lead to urine leakage. Also, when the absorbing fiber is too low, the ADS may not provide sufficient capillarity to drain fluid from a topsheet.

The quantitative chemical composition of nonwoven comprising a mixture of fiber types is determined using ISO 1833-1.

Any suitable absorbing materials for the absorbing fibers may be utilized. Some examples of absorbing materials include cotton, pulp, rayon or regenerated cellulose or combinations thereof. In one example, the first layer may comprise viscose cellulose fibers. The length of the absorbing fibers can be in the range of about 20 mm to about 100 mm, or about 30 mm to about 50 mm or about 35 mm to about 45 mm, specifically reciting all values within these ranges and any ranges created thereby. In general, the fiber length of pulp is from about 4 to 6 mm and is not proper used in conventional carding machines because the pulp fibers are too short. Therefore, if pulp is desired as a fiber in the first layer, then additional processing to add pulp to the nonwoven webs may be required. As an example, pulp may be airlaid between nonwoven webs with the combination being subsequently integrated. As another example, tissue may be utilized in combination with the nonwoven webs and the combination may be subsequently integrated.

A wide variety of polymers may be used for the synthetic fibers. Examples of suitable synthetic fibers include polyolefins such as polypropylene and polyethylene, and copolymers thereof, polyesters such as polyethylene terephthalate (PET), polytrimethyene terephthalate (PTT), and polybutylene terephthalate (PBT), nylons, polystyrenes, copolymer or blends thereof, and other synthetic polymers conventional in the preparation of fibers. The synthetic fibers may be staple thermoplastic fibers.

Suitable materials for the thermoplastic fibers include monocomponent or multicomponent fibers, or mixtures thereof. Suitable monocomponent thermoplastic fibers may include PET. The thermoplastic fiber may comprise a sheath/core bicomponent fiber. The sheath/core bicomponent fiber may comprises a sheath comprising a polymer having a lower melting temperature than that of a polymer forming the core. The lower melting polymer of the sheath may promote bonding while the higher melting polymer of the core may provide strength to the thermoplastic fiber and thus to the first layer. The thermoplastic fibers typically have lengths reigning from about 15-100 mm, or about 20-100 mm, or about 3-15 mm. In some embodiments, the sheath/core bicomponent fibers may comprises PE/PET fibers, PE/PP fibers or a mixture thereof. In some embodiments, the thermoplastic fibers are made by sustainable polymers such as polymers derived from a bio-based material. Sustainable polymers may include polylactic acid and bio-based polyethylene.

The synthetic fibers may have a dtex of between about 0.3 dtex to about 15 dtex, or from about 0.5 dtex to about 10 dtex, or from about 0.5 dtex to about 6 dtex.

The first layer may further comprise a binder such as latex. The binder may function as an auxiliary, for immobilize or entrap the absorbing fibers.

The first layer may be substantially free of superabsorbent polymer. When a material is substantially free of superabsorbent polymer in the disclosure herein, it intends to mean the material contains superabsorbent polymer less than about 5%, or about 2%, or about 1%, or 0% by the total weight of the material containing superabsorbent polymer. The first layer may include superabsorbent polymer.

The first layer comprises at least two stratums, the at least two stratums are integrated without adhesives.

Referring to FIG. 3 the first layer comprises a first stratum forming the first surface 15 and a second stratum forming the second surface 16. The first layer may further comprise one or more additional stratums deposited between the first stratum and the second stratum.

The first stratum comprises a different fiber blend than the second stratum. The first stratum comprises first absorbing fibers, and a second stratum comprises second absorbing fibers. The percentage of the second absorbing fibers in the second stratum is at least about 15% higher than, or at least about 17% higher than, or at least 19% higher than the percentage of the first absorbing fibers in the first stratum as measured according to the Fiber Number Test. Such configuration of having higher percentage of the second absorbing fibers in the second stratum than the percentage of the first absorbing fibers in the first stratum may enhance a fluid absorption speed and/or reduce rewet of absorbent articles.

In one embodiment, assuming the first stratum would be closest to the wearer-facing surface in an absorbent article, the fiber selection for a first stratum may be such that there is more openness associated with this web. A second stratum may be similarly configured, or may be configured to collect liquid insults from the void space of the first stratums and effectively distribute these liquid insults to an absorbent core.

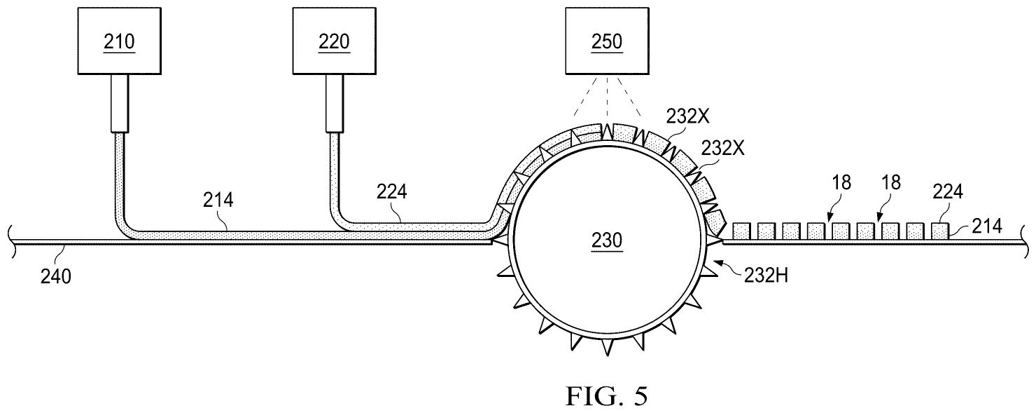

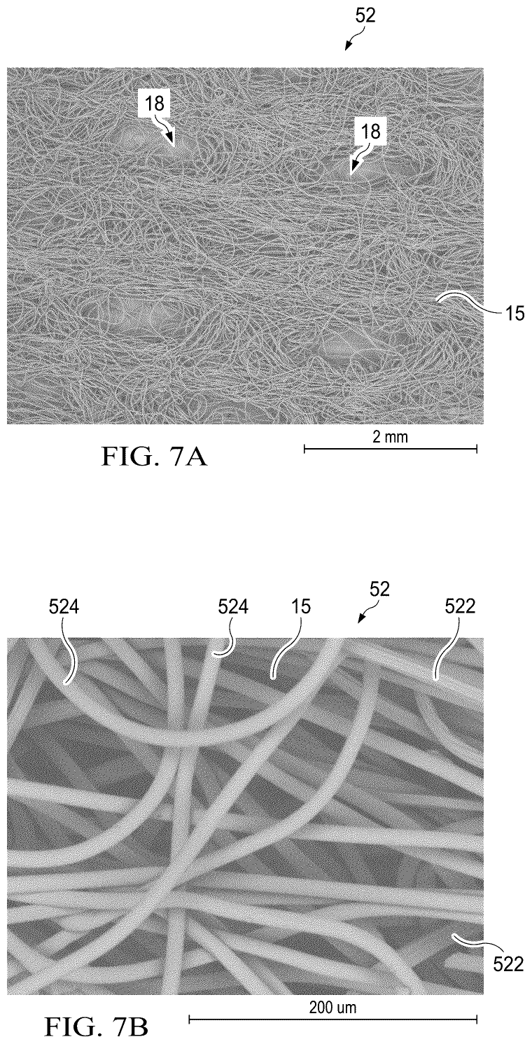

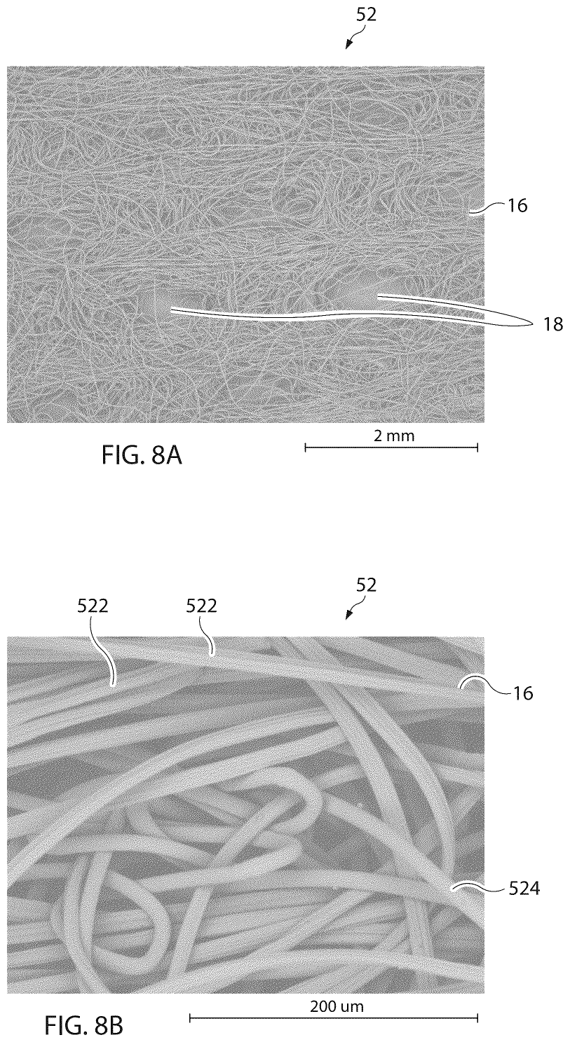

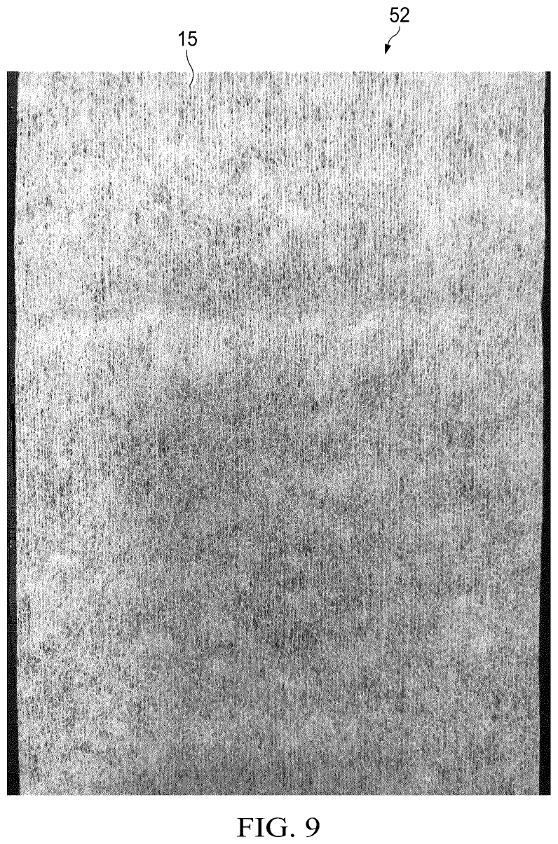

An exemplary first layer of the ADS according to the present invention is shown in the scanning electron microscope images of FIGS. 6-8B. FIG. 6 shows a first surface or a top surface 15 of a first layer 52 in the ADS 50 suitable for the present invention. The first layer 52 comprises a plurality of apertures 18. Referring to FIGS. 3 and 4, the first layer 52 has a first stratum 320 forming the first surface 15, and a second stratum 340 forming the second surface or bottom surface 16, and a plurality of apertures 18. FIG. 7A is SEM image of a first surface 15 of a first layer 52 of the ADS 50 suitable for the present invention, and FIG. 7B is an enlarged SEM image of a first surface 15 of a first layer 52 of the ADS 50. The first stratum of the first layer 52 forms the first surface 15. FIG. 8A is SEM image of a second surface 16 of the first layer 52 of the ADS 50 suitable for the present invention, and FIG. 8B is an enlarged SEM image of a second surface 16 of a first layer 52 of the ADS 50. The second stratum of the first layer 52 forms the second surface 16.

Fibers of the stratum may be integrated. Where at least two stratums are utilized, fibers in each stratum of the at least two stratums may be integrated. Where at least two stratums are utilized, fibers of the at least two webs may be integrated.

When the stratum(s) are integrated, they cannot be manually separated—at least not without substantial effort and time. Each stratum forms a stratum in the overall ADS. Each stratum can maintain its unique properties for at least a portion of the stratum along the z-direction, even when integrated into a larger ADS. The ADS can provide capillary suction to “pull” fluid through the topsheet, which is competing for trickle/low flow conditions. The ADS also can contain a gush by providing distribution functions to efficiently utilize the absorbent core, as well as provide intermediate storage until the absorbent core can accept fluid.

The first layer may have a basis weight in the range of about 15-140 gsm, or about 30-120 gsm, or about 40-80 gsm. A basis weight of the first layer may be determined to balance acquisition-distribution performance and a thickness of the absorbent article.

The first layer web may comprise a carded web, air-laid web, wet-laid web, and the like. In some embodiments, the first layer comprises a spunlace web.

The first layer comprises a plurality of apertures. With the first layer comprising a plurality of apertures, the ADS, eventually the absorbent article may provide a consistent absorption speed even upon repeated gushes, and mitigate reducing of air permeability in a wet condition. Further, with the first layer comprising a plurality of apertures, the first layer may have an improved softness represented by a MD stiffness and a CD stiffness as measured according to the Softness Test disclosed herein. The apertures in the first layer may open from a first surface 15 of the first layer and to a second surface 16 of the first layer 52, so that the apertures are open in a thickness direction of the first layer. The apertures in the first layer may open either in the first surface 15 of the first layer or the second surface 16 of the first layer 52.

With such first layer nonwoven configuration that having the percentage of the second absorbent fibers in the second stratum higher than the percentage of the first absorbent fibers in the first stratum, and a plurality of apertures, an absorbent article comprising an ADS comprising the first layer surprising has a low run-off of liquid.

The aperture may be in any shape, for example, preferably, a circular shape, a semicircular shape, an elliptical shape, a polygonal shape, and a slit shape such as a straight line shape or a curved line shape. The aperture may have a length from about 100 μm to about 5000 μm, or from about 150 μm to about 2000 μm, or from about 200 μm to about 3000 μm. When the aperture is too small such as having an aperture length lower than 100 μm, the liquid absorption speed may get slower. When the aperture is too big such as having a width higher than 5000 μm, the rewet may increase.

The first layer may comprise apertures in the range of about 10-200 numbers/cm², or about 20-80 numbers/cm².

The first layer may comprise a wet air permeability no lower than about 15 m³/m²/min, or no lower than about 25 m³/m²/min, or no lower than about 30 m³/m²/min as measured according to Air Permeability Test. The first layer may comprise a ratio of a wet air permeability to a dry air permeability no lower than about 10%, or no lower than about 13%, or no lower than about 15% when the wet air permeability and the dry air permeability are measured according to Air Permeability Test.

The first layer may comprise a vertical wicking height no lower than about 40 mm, or no lower than about 45 mm, or no lower than about 47 mm as measured according to Vertical Wicking Height Test.

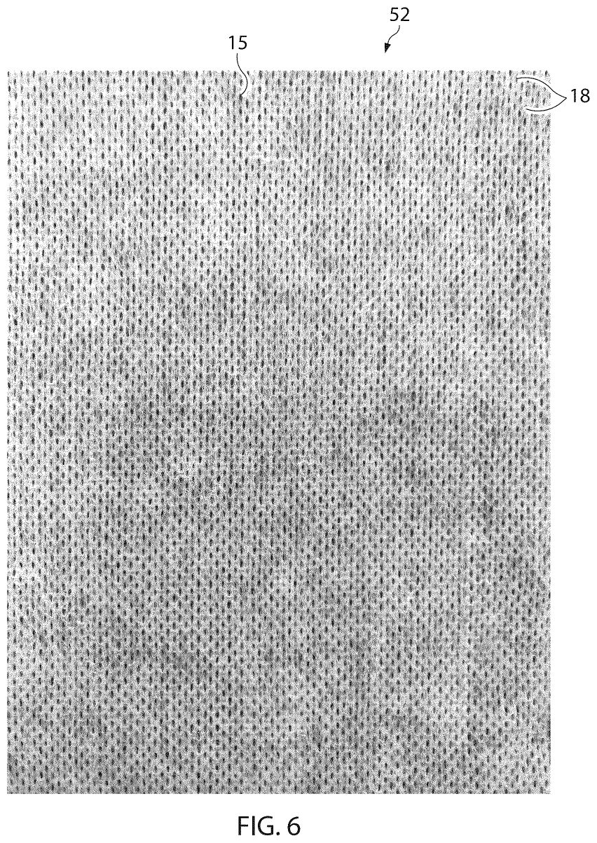

An exemplary process for forming the fluid management layer of the present disclosure is shown in FIG. 5. Referring to FIG. 5, a plurality of carding machines 210 and 220 may each create a nonwoven web (stratum), e.g. 214 and 224, respectively, which is transferred to a carrier belt 240. It is also worth noting that after the nonwoven web 214 is deposited on the carrier belt 240, the nonwoven web 224 is then deposited on the first nonwoven 214 on the carrier belt 240. Subsequently, each of the first and second nonwoven webs 214 and 224 are then provided to an integration process 250, together with the receiving roll 230, which utilizes high-pressure water streams to entangle the fibers of the first and second nonwoven webs. Both carding and integration processes are well known in the art. The receiving roll 230 comprises a plurality of protrusions 232X, and mesh 232H for drainage of water. Due to protrusions 232X on the receiving roll 230 fibers are pushed around the protrusions 232X to form apertures in the spunlace process directly. With adjustment of a height of the protrusions 232X over the thickness of nonwoven web, the apertures may completely pass through the nonwoven web in the thickness direction of the nonwoven, or they may not completely pass through the nonwoven web in the thickness direction of the nonwoven.

It is worth noting that with the arrangement provided in schematic diagram of FIG. 5, a wide variety of configurations for a first layer nonwoven may be achieved. However, it is important that the ADS of the present disclosure have adequate openness to allow for quick acquisition of fluid yet also are able to lock away liquid insults to reduce the likelihood of rewet. With this in mind, the nonwoven webs, i.e. 214 and/or 224, are different from one another in fiber compositions. Assuming the first nonwoven web would be closest to the wearer-facing surface in an absorbent article, the percentage of absorbing fibers in the second nonwoven web (stratum) 214 is at least 15% higher than the percentage of absorbing fibers in the first nonwoven web (stratum) 224. The fiber selection for the first nonwoven web 214 may be such that there is more openness associated with this web. The second nonwoven web 224 may be configured collect liquid insults from the void space of the first nonwoven webs 214 and effectively distribute these liquid insults to an absorbent core.

### Second Layer

Referring to FIG. 4, ADS 50 according to the present invention may further comprise a second layer 54 having a top surface 21 and a bottom surface 22 which is positioned on top of the first layer 52 in such a way that the top surface 15 of the first layer 52 and the bottom surface 22 of the second layer 52 are faced to each other. The second layer 54 comprises second thermoplastic fibers. Examples of the second thermoplastic fibers for the second layer include thermoplastic fibers discussed with respect to the first layer. The second thermoplastic fiber may or may not be the same as the first thermoplastic fiber.

The second layer may be free of absorbing fibers.

The second layer may have a basis weight in the range of about 15-80 gsm, or about 30-70 gsm, or about 40-60 gsm.

In some embodiments, the second layer comprises a carded nonwoven. In such embodiments, the second layer may comprise an air-through bonding nonwoven.

An ADS for the present invention may be substantially free of superabsorbent material.

An ADS for the present invention may have a basis weight in the range of about 20-220 gsm, or about 40-160 gsm, or about 20-140 gsm, or about 40-80 gsm. A basis weight of the first layer may be determined to balance acquisition-distribution performance and a thickness of the absorbent article.

An ADS suitable for absorbent articles according to the present invention may be manufactured via various process known in the industry.

An ADS suitable for the present invention, when consisting of a first layer comprising first thermoplastic fibers and absorbing fibers, can be formed for example by air-laying a mixture of the first thermoplastic fibers and absorbing fibers directly onto a carrier wire to form a web, and subsequently subjecting the web to compression and/or thermal treatment in order to bind at least a portion of the first thermoplastic fibers.

When the ADS suitable for the present invention comprises a first layer comprising first thermoplastic fibers and absorbing fibers and a second layer comprising second thermoplastic fibers, as one example, a web for the ADS may be manufactured by a process comprising the steps of forming a first layer web comprising first thermoplastic fibers and absorbing fibers, forming a second layer web comprising second thermoplastic fibers, forming a composite web by overlaying the first layer web on the second layer web or vice versa, and subjecting the composite web to compression, adhesive, and/or thermal treatment in order to bond at least portion of the first and the second thermoplastic fibers.

As another example, a composite nonwoven for the ADS may be manufactured in a continuous process. For example, the process may comprise the steps of a) supplying a second layer web comprising second thermoplastic fibers, b) overlaying a first layer web comprising first thermoplastic fibers and absorbing fibers on one surface of the second web to form a composite web, and c) subjecting the composite web to compression and/or thermal treatment in order to bond at least portion of the first and the second thermoplastic fibers. In the example, in step a), the second layer web may be supplied from a spool on which a previously formed second layer web is wounded. Or, the second layer web may be supplied by preparing the second layer web using a web forming device, for example, a card or spinning beam. The second layer web may be supplied using an air-through bonded nonwoven forming device.

Step b) may be conducted by air-laid process using at least one forming head. For example, in one or more forming heads a stream of the first thermoplastic fibers and absorbing fibers are homogeneously mixed to form a stream of mixed fibers, and each forming head deposits the stream of mixed fibers on to one surface of the second layer web. In step c), the compression may be carried out using one or more pairs of compaction rollers that are disposed following the one or more forming heads. When present, compaction rollers may be heated at a temperature, for example ranging from about 90 to 110° C. In step c), thermal treatment can be conducted using any conventionally known thermal treatment method. Examples of preferable treating process include a thermal treatment apparatus such as a hot air through-type thermal treatment apparatus, a hot air blowing thermal treatment apparatus, an infrared thermal treatment apparatus, or the like. These thermal treatment apparatuses are typically provided with a conveying support for supporting and conveying a web.

An ADS suitable for the present invention, when comprising the first layer and a second layer, the first layer may have a fiber density higher than the second layer.

The ADS for the present invention may further comprise one or more additional layers deposited on the outer surface 16 of the first layer 52.

The absorbent article of the present invention comprises a liquid pervious topsheet, a liquid impervious backsheet, an absorbent core disposed between the topsheet and the backsheet, and an ADS disclosed herein disposed between the topsheet and the absorbent core.

Components of the disposable absorbent article described in this specification can at least partially be comprised of bio-sourced content as described in US 2007/0219521A1 Hird et al published on Sep. 20, 2007, US 2011/0139658A1 Hird et al published on Jun. 16, 2011, US 2011/0139657A1 Hird et al published on Jun. 16, 2011, US 2011/0152812A1 Hird et al published on Jun. 23, 2011, US 2011/0139662A1 Hird et al published on Jun. 16, 2011, and US 2011/0139659A1 Hird et al published on Jun. 16, 2011. These components include, but are not limited to, topsheet nonwovens, backsheet films, backsheet nonwovens, side panel nonwovens, barrier leg cuff nonwovens, super absorbent, nonwoven acquisition layers, core wrap nonwovens, adhesives, fastener hooks, and fastener landing zone nonwovens and film bases. In at least one embodiment, a disposable absorbent article component comprises a bio-based content value from about 10% to about 100% using ASTM D6866-10, method B, in another embodiment, from about 25% to about 75%, and in yet another embodiment, from about 50% to about 60% using ASTM D6866-10, method B. In order to apply the methodology of ASTM D6866-10 to determine the bio-based content of any disposable absorbent article component, a representative sample of the disposable absorbent article component must be obtained for testing. In at least one embodiment, the disposable absorbent article component can be ground into particulates less than about 20 mesh using known grinding methods (e.g., Wiley® mill), and a representative sample of suitable mass taken from the randomly mixed particles.

In some embodiments, the absorbent article of the present invention comprises at least one of a topsheet and a backsheet comprising a natural fiber.

In some embodiments, the absorbent article of the present invention comprises an ADS disclosed herein wherein the first layer has a fiber density higher than the second layer.

When the ADS of the present invention comprises the first layer and the second layer, the ADS is disposed in the absorbent article in such a way that the second layer is disposed between the topsheet and the first layer.

In one embodiment, the absorbent article of the present invention comprises an absorbent core comprising at least about 70%, at least about 80%, about 85%, about 90% or about 95% of superabsorbent polymers by weight of the absorbent core.

In another embodiment the absorbent article of the present invention comprises at least one of components such as a topsheet, an absorbent core, an ADS and a backsheet of the absorbent article comprises natural fibers.

The ADS disclosed herein may be mechanically deformed. The ADS may comprise a plurality of protrusions extending outwardly from at least one surface of the ADS and the protrusions extending outwardly are oriented towards the absorbent core of the absorbent article.

Deformed ADS may improve its mechanical properties such as flexibility and cushiness which are considered trade-offs.

### Absorbent Article Manufacturing Process

The absorbent articles of the invention may be made by any conventional methods known in the art. In particular the articles may be hand-made or industrially produced at high speed. Typically, adjacent layers and components will be joined together using conventional bonding method such as adhesive coating via slot coating or spraying on the whole or part of the surface of the layer, or thermo-bonding, or pressure bonding or combinations thereof. Other glues or attachments are not represented for clarity and readability but typical bonding between the layers of the article should be considered to be present unless specifically excluded. Adhesives may be typically used to improve the adhesion of the different layers, for example between the backsheet and the core wrap. The glues used may be any standard hotmelt glue as known in the art.

### Nonwoven

The present invention also relates to a nonwoven comprising a first layer comprising a first stratum and a second stratum, the first stratum comprising first absorbing fibers, and a second stratum comprising second absorbing fibers, wherein the percentage of the second absorbing fibers in the second stratum is at least about 15% higher than the percentage of the first absorbing fibers in the first stratum as measured according to the Fiber Number Test. The nonwoven of the present invention is suitable for an acquisition-distribution system in an absorbent article. When the nonwoven of the present invention is used as a component layer such as ADS in an absorbent article, the nonwoven is disposed in such a way that the first surface of the nonwoven faces a topsheet side, and the second surface of the nonwoven faces an absorbent core side.

The first layer may have a vertical wicking height no lower than about 40 mm, or no lower than about 45 mm, or no lower than about 48 mm as measured according to Vertical Wicking Height Test. The first layer may have a wet air permeability no lower than about 15 m³/m²/min, or no lower than about 25 m³/m²/min, or no lower than about 30 m³/m²/min as measured according to Air Permeability Test. The first layer has a ratio of a wet air permeability to a dry air permeability no lower than about 10%, when the wet air permeability and the dry air permeability are measured according to Air Permeability Test. The nonwoven may further comprise a second layer comprising thermoplastic fibers in such a way the second layer is positioned above the first layer. Descriptions under the first layer and second layer of ADS disclosed above are applicable for the nonwoven of the present invention.

Having a nonwoven of the present invention as an ADS in an absorbent article, the absorbent article may have a low run-off of liquid. Therefore, the risk of leakage of the absorbent article is reduced when the nonwoven disclosed herein invention is used in an absorbent article.

### Measurements

Unless particularly mentioned, all tests are performed in a room maintained at 23±2° C. and 50±5% relative humidity, and samples are pre-conditioned in a room maintained at about 23±2° C. and about 50±5% relative humidity, for at least 2 hours prior to testing.

### 1. Fiber Number Test

If a nonwoven is available in its raw material form, a specimen is cut from the raw material. If a nonwoven is a component layer of an absorbent article, the absorbent article is cut and the nonwoven layer is removed from the absorbent article, using a razor blade to excise the nonwoven layer from the underling layers of the absorbent article. Any remaining adhesive may be removed from the specimen using Tetrahydrofuran (THF) as solvent.

Scanning electron microscope (“SEM”) images of a first surface and an opposite second surface of nonwoven specimen are taken by a scanning electron microscopy, Hitachi TM300 or equivalent. When the nonwoven is a component layer of an absorbent article, the first surface of the nonwoven specimen is a surface facing topsheet side, and the second surface of the nonwoven is surface facing an absorbent core side. 8 SEM images of a test nonwoven specimen are taken.

The nonwoven specimen is placed on the microscope stage using double-sided conductive tape or clapped to fix the specimen. An appropriate magnification can be chosen such that features in the nonwoven specimen are suitably clear and enlarged for measurement.

In each SEM image, select an area of a width 240 μm and a length 330 μm to count numbers of absorbing fibers and total fibers. Count numbers of absorbing fibers such as viscose fibers, and all other fibers such as and synthetic fibers to obtain numbers of total fibers. The absorbing fibers may be judged based on morphology difference from synthetic fibers. For each nonwoven specimen, 8 images are tested. Total number of absorbing fibers from the 8 images is reported as number of absorbing fibers. Total number of all fibers from the 8 images is reported as total number of fibers. Number of absorbing fibers and number of total fibers obtained from the first surface images are reported as number of first absorbing fibers and number of total fibers in the first stratum, respectively. Number of absorbing fibers and number of total fibers obtained from the second surface images are reported as number of first absorbing fibers and number of total fibers in the second stratum, respectively.

Percentage of absorbent fibers in the first stratum is calculated by dividing number of the absorption fibers by number of total fibers in the first stratum. Percentage of absorbent fibers in the second stratum is calculated by dividing number of the absorption fibers by number of total fibers in the second stratum.

### 2. Aperture Dimension Test

### 2-1 Aperture Size

Place a nonwoven specimen on a black plate of a microscope such as VHX-5000 (Keyence) or equivalent. Using the microscopy, obtain a microscopic image at magnification of 50× and add grids with a grid size of 100 μm×100 μm on the microscopic image. The image has a size about 700 mm×500 mm. Upload the microscopic image on Microsoft PowerPoint and adjust the microscopic image contrast to 100%.

Determine minimum rectangles consisting of intact girds to include a black area to calculate aperture sizes. A black area whose length or width is not less than 100 μm is counted as an aperture. Two adjacent black areas having the shortest distance therebetween less than 100 μm is counted as one aperture. Measure lengths and widths of at least 30 rectangles. Averages of the length and the widths form the 30 rectangles are reported as a length of aperture and a width of aperture, respectively.

### 2-2 Aperture Density

Place a nonwoven specimen on a flat black background. Place a transparent grid template with a grid size of 1 cm×1 cm on top of the nonwoven specimen. A microscopic image is taken by a microscopy such as VHX-5000 (Keyence) or equivalent at magnification of 2×. Then, count number of intact apertures in each of 8 grids. The average number of apertures is reported as density of apertures.

### 3. Vertical Wick Height Test

In order to measure weaking power of a nonwoven, a vertical wicking height of the nonwoven is measured according to INDA/EDANA STANDARD TEST: WSP 10.1 (05) “7.3 The liquid wicking rate (capillarity))”. The wicking height is recorded at 600 s. At least 8 replicas for a test nonwoven are tested. The average value of the 8 replicas is reported as a vertical wicking height.

### 4. Air Permeability Test

Air permeabilities of an original nonwoven and a wet nonwoven are tested. A wet sample is prepared as follows:

Place the above dry sample nonwoven into a basin with sufficient distilled water so that the fluid completely covers the sample nonwoven. Submerge the sample nonwoven in the water and stay for 5 min for the sample nonwoven to completely absorb the water. Take out the sample nonwoven using a holder from the water and stay the nonwoven vertically in 20 min to remove free water from the nonwoven.

All measurements are performed in a laboratory maintained at 23±2° C. and 50±2% relative humidity and test specimens are conditioned in this environment for at least 2 hours prior to testing.

The air permeability of a nonwoven is tested according to INDA/EDANA Nonwovens Standard Procedures NWSP 070.1.R0 (15) making use of a Textest FX3300 (Textest Instruments, Schwerzenbach, Switzerland) air permeability tester or equivalent. A circular test head with an area of 38 cm² is used, and while a fixed pressure of 125 Pa is maintained across a nonwoven specimen, air flow through the specimen is measured in cubic meter per square meter per minute (m³/m²/min). 5 rectangular specimens are taken such that each specimen center corresponds to the center of the material and such that the length and width of each specimen are greater than the smallest dimension of the circular head. the air permeability of each is recorded in m³/m²/min to the nearest 1 m³/m²/min. The arithmetic mean of the individual specimen results is calculated and reported as the air permeability in units m³/m²/min to the nearest 1 m³/m²/min.

### 5. Softness Test

To measure softness of a nonwoven, a MD stiffness and a CD stiffness of the nonwoven are tested by the INDA IST 90-3 standard test method for Handle-O-meter stiffness of NWs.

### 6. Run-Off Test

Run-off is measured according to a method, a modified method for testing nonwovens in WSP 80.9 (05), standard test method for nonwoven run-off. The inclination angle is set to be 300+/−1°. A total mass of test liquid of 75±0.5 g is used.

Put the diaper on a plate. Place one end of the plate with diaper front side higher than the end with diaper back side. The angle formed between the plate and the horizontal line is around 30° degree. Set the 10 cm from the top edge of the absorbent core in the diaper as loading point and release 75 g of a saline solution (0.9% NaCl) at the loading point. Collect the saline solution which runs off the surface of the diaper below the plate. Measure the weight of the run-off fluid in the plate.

Run-off (%)=(Weight (g) of run-off fluid/75 g)×100

### 7. Modified Fluid Acquisition Test

The Modified Fluid Acquisition (“MFA”) Test is designed to measure the speed at which 0.9% saline solution is absorbed into an absorbent article that is compressed at 2.07 kPa. A known volume is introduced four times, each successive dose starting five (5) minutes after the previous dose has absorbed. Times needed to absorb each dose are recorded. All testing is performed in a room also maintained at about 23° C.±2° C. and about 50%±2% relative humidity. The test fluid is 0.9% w/v saline solution and is prepared by weighing 9.0 g±0.05 g of NaCl into a weigh boat, transferring it into a 1 L volumetric flask, and diluting to volume with de-ionized water.

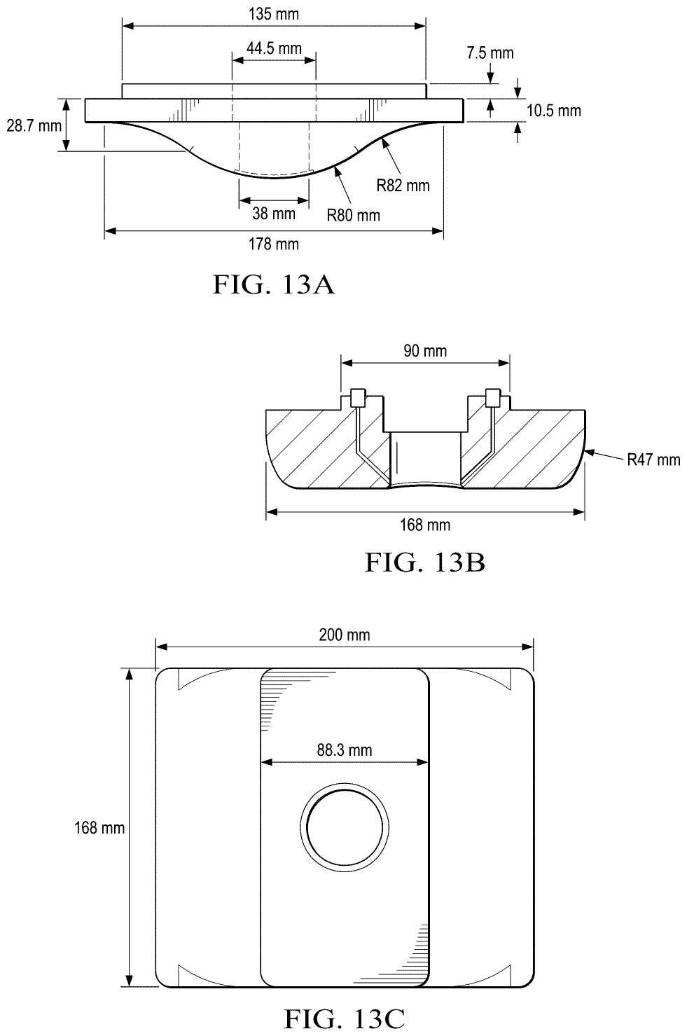

The MFA apparatus is depicted in FIG. 12 through FIG. 14B. The MFA apparatus comprises a bladder assembly 3001 and a top plate assembly 3200 that includes a deposition assembly 3100. A controller 3005 is used to 1) monitor the impedance across electrodes 3106, recording the time interval 0.9% saline solution is in a cylinder 3102, 2) interface with a liquid pump 3004 to start/stop dispensing, and 3) time intervals between dosing. The controller 3005 is capable of recording time events to ±0.01 sec. A house air supply 3014 is connected to a pressure regulator 3006 capable of delivering air at a suitable flow/pressure to maintain 2.07 kPa in the bladder assembly 3001. A liquid pump 3004 (Ismatec MCP-Z gear pump, available from Cole Palmer, Vernon Hills, IL or equivalent) capable of delivering a flow of 10-80 mL at a rate of 3-15 mL/s is attached to a steel tube 3104 of the deposition assembly 3100 via tygon tubing 3015.

The bladder assembly 3001 is constructed of 12.7 mm Plexiglas with an overall dimension of 80 cm long by 30 cm wide by 10 cm tall. A manometer 3007 to measure the pressure inside the assembly and a pressure gauge 3006 to regulate the introduction of air into the assembly are installed through two holes through the right side. A bladder 3013 is assembled by draping a 50 mm by 100 mm piece of silicone film, (thickness 0.02″, Shore A durometer value of 20, available as Part #86435K85 from McMaster-Carr, Cleveland, OH) over the top of the box with enough slack that the film touches the bottom of the box at its center point. An aluminum frame 3003 with a flange is fitted over the top of the film and secured in place using mechanical clamps 3010. When in place, the assembly should be leak free at a pressure of 3.45 kPa. A front 3008 and back 3009 sample support 5 cm by 30 cm by 1 mm are used to anchor the sample. The absorbent article is attached to the top surface of the sample supports by either adhesive tape or mechanical “hook” fasteners. These supports can be adjusted along the length of the aluminum frame 3003 via a simple pin and hole system to accommodate different size absorbent articles and to correctly align their loading point.

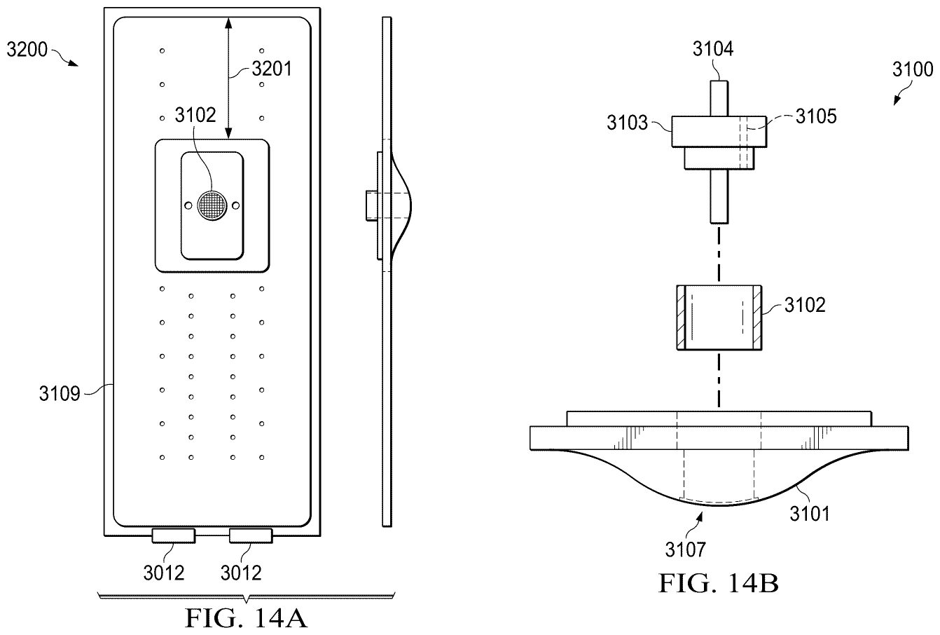

The top plate assembly 3200 is constructed of an 80 cm by 30 cm piece of 12.7 mm Plexiglas reinforced with an aluminum frame 3109 to enhance rigidity. The plate has a cutout 170 mm wide by 201 mm long centered laterally on the plate, 170 mm from the front of the plate 3201 for mounting of the deposition assembly. In addition, the top plate has thirty-six (36) 3.2 mm diameter holes drilled through it distributed as shown in FIG. 14A. The holes prevent air from being trapped under the top plate as the bladder is inflated. The top plate assembly 3200 is connected to the bladder assembly 3001 via two hinges 3012. During use, the top assembly is closed onto the bladder assembly and locked into place using a mechanical clamp 3011.

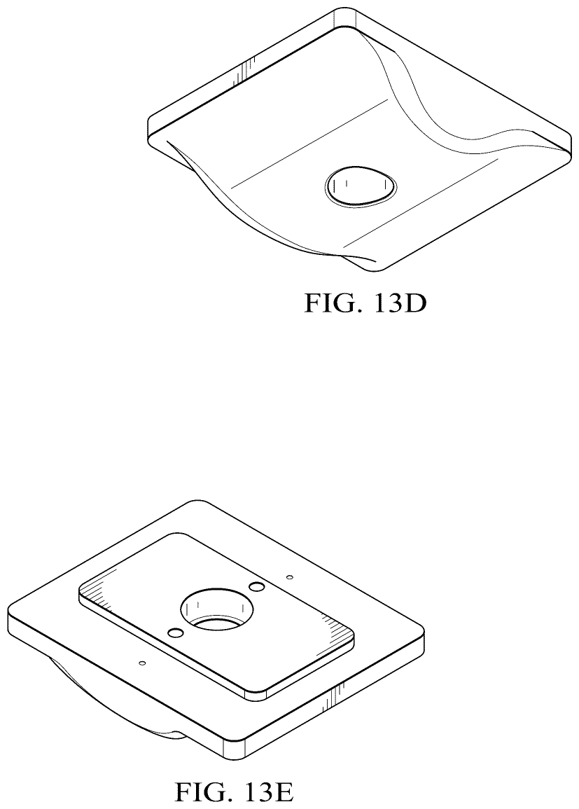

The deposition assembly 3100 is fitted into the top plate 3200 and includes 1) a liquid introduction cylinder 3102, 2) a curved surface 3101 at the loading point of the absorbent article and 3) electrodes 3106 that are used to detect fluid in the cylinder 3102. The detailed dimensions of the curved component are provided in FIG. 13A to FIG. 13E. FIG. 13A is a side view of the curved component. FIG. 13B is an end view of the curved component. FIG. 13C is a bottom view of the curved component. FIG. 13D is a bottom perspective view of the curved component. FIG. 13E is a top perspective view of the curved component. This curved component can be milled or 3D printed. The top portion of the introduction cylinder is a 50.8 mm O.D. Plexiglas cylinder 3102 with a 38.1 mm I.D. This is fitted into the curved component to give the introduction cylinder a total height of 100 mm. Imbedded electrodes run from connectors on the upper surface of the curved component and terminate flush with an inside wall of the introduction cylinder, 2 mm from the bottom of the cylinder. The two electrodes are positioned 180 degrees apart. A nylon screen 3107 is cut and affixed flush with the bottom of the cylinder such that the sample cannot swell into the cylinder. A 5 mm semi-circle is cut in the screen in the immediate area of the two electrodes. The deposition assembly is inserted into the top plate as shown in FIG. 14A such that the curved surface is flush with the bottom of the top-plate assembly 3200. The introduction cylinder 3102 is topped with a loose-fitting nylon cap 3103. The cap has a 6.35 mm O.D. steel tube 3104 inserted through its center. When the cap is in place, the bottom of the tube ends 20 mm above the screen 3107. The cap also has an air hole 3105 to ensure negative pressure does not impede the absorption speed.

All sample articles are conditioned at 23° C.±2° C. and about 50%±2% relative humidity for two hours prior to testing. The absorbent article is first prepared by excising any inner or outer leg cuffs, waist caps, elastic ears or side panels, taking care not to disturb the top sheet that resides above the article's core region. Place the absorbent article flat onto a lab bench and identify the intersection of the longitudinal centerline with the size dependent loading point (as defined in Table 1).

TABLE 1
Loading Points, Volumes, and Flow rate for Acquisition Testing:

| | | Loading Point from front of | | | |
|---|---|---|---|---|---|
| | Approximate | Core* | | Single Dose | Flow |
| Diaper | Baby Weight | Boy | Girl | Volume | Rate |
| Size | Pounds | mm | mm | mL | mL/s |
| 1 | 8 to 13 | 64 | 64 | 24 | 8 |
| 2 | 13 to 17 | 76 | 89 | 24 | 8 |
| 3 | 17 to 28 | 89 | 114 | 50 | 10 |
| 4-6 | 28+ | 102 | 127 | 75 | 15 |

*The boy loading point is used for unisex diapers.

Attach the front end of the absorbent article to the top surface of the front sample plate 3008 by either adhesive tape or mechanical “hook” fasteners with the top sheet facing upward. The placement is such that just the chassis and not the absorptive core overlays the plate. The sample plate 3008 is attached to the aluminum frame 3003 such that the size-dependent Loading Point (as defined in Table 2) of the absorbent article will be centered longitudinally and laterally within the cylinder 3102 when the top plate assembly has been closed. The back end of the absorbent article is secured to the back sample plate 3009 by either adhesive tape or mechanical “hook” fasteners, once again ensuring that only the chassis and not the absorptive core overlays the plate. The back sample plate 3009 is then attached to the aluminum frame 3003 such that the article is taunt but not stretched. The top plate assembly is closed and fastened, and the bladder is inflated to 2.07 kPa±0.07 kPa. The pressure is maintained at this level during the complete loading sequence of the test.

The pump 3004 is primed and then calibrated to deliver the size-dependent volume and flow rate selected from Table 2. Volume and flow rate must be within ±2% of target. The cap 3103 is placed into the cylinder 3102. The controller 3005 is started, which in turn delivers the first dose of 0.9% saline solution. After the volume has been absorbed, the controller waits for 5.0 minutes before addition of the next dose. This cycle is repeated for a total of four doses. If the fluid leaks out of or around the article (i.e., is not absorbed into the article) then the test is aborted. Also if any acquisition time exceeds 1200 seconds, the test is aborted. The acquisition time is defined as the difference between the start time (i.e., when the 0.9% saline is first introduced into the cylinder and that conducting fluid completes the circuit between the electrodes) and the stop time (i.e., when the fluid has completely drained from the cylinder and the circuit between the electrodes is broken). Acquisition times are recorded by the controller for each dose to the nearest 0.01 second. After the last dose is acquired, pressure is applied for an additional 10 minutes. Open the pressure relief valve 3016 to deflate the bladder and then remove the sample from the acquisition system.

In like fashion, run a total of five (5) replicates for each absorbent article to be evaluated. Calculate and report the Acquisition Times (sec) for each dose as the arithmetic mean of the replicates to the nearest 0.01 sec.

### 8. Light Touch Dryness Test

The Light Touch Dryness (“LTD”) Test is performed immediately after the MFA Test. The test comprises measuring the mass of fluid expressed from the absorbent article under pressure after loading by the MFA protocol. Whatman #1 filter paper sheets are used as the rewet substrate and are conditioned at 23° C.±2° C. and about 50%±2% relative humidity overnight before use. All testing is performed in a room also maintained at about 23° C.±2° C. and about 50%±2% relative humidity.

Equipment for this test includes a Plexiglas disk 70.0 mm in diameter and 20 mm thick and a stainless steel confining weight that rests upon it. The mass of the disk and confining weight combined is 812 g±2 g which corresponds to a pressure of 2.07 kPa. Whatman #1 filter paper is die cut into 70.0 mm diameter circles and stacks of four (4) assembled for use during rewet testing. Measure and record the mass of the dry filter paper stack and record to the nearest 0.0001 g.

Within 30 seconds after the conclusion of the MFA test, remove the absorbent article from the acquisition apparatus and place flat on a bench top with the top sheet facing upward. Place a pre-weighed stack of Whatman #1 filter paper centered at the loading point (as determined previously in the MFA test), place the Plexiglass disk onto the stack, and gently place the confining weight onto the disk. Wait for 30.0 sec±0.5 sec and remove the weight and disc. Immediately measure the mass of the wet filter paper and record to the nearest 0.0001 g. Calculate the modified rewet value as the difference between the wet and dry weight of the stack and record to the nearest 0.1 mg.

In like fashion run a total of five (5) replicates for each absorbent article to be evaluated. Calculate and report the Light Touch Dryness (mg) for each dose as the arithmetic mean of the replicates to the nearest 0.1 mg.

### EXAMPLES

### Example 1. Nonwoven Preparation

Nonwoven substrates were produced according to Table 2. All the nonwoven substrates were hydroentangled, spunlaced. Nonwoven 1 and Nonwoven 3 were produced using hydroentanglement spunlace process represented in FIG. 5.

FIGS. 6-8B are SEM images of Nonwoven 1. Referring to FIGS. 6 and 7A, Nonwoven 1 contains a plurality of apertures 18. Nonwoven 1 comprises a first stratum and second stratum, the first stratum forming the first surface 15 of Nonwoven 1, and the second stratum forming the second surface 16 of Nonwoven 1. Each of the first stratum and the second stratum comprises absorbing fibers, 1.7 dtex viscose in this case, and synthetic fibers, 1.56 dtex PET fibers in this case. The absorbing fibers and the synthetic fibers are differentiated based on morphology difference. Viscose fibers have a surface with grooves along a fiber length direction while PET fibers have a smooth surface. FIG. 7B is an enlarged SEM image of a first surface 15 of Nonwoven 1 which shows the absorbing fibers and synthetic fibers more clearly. FIG. 8B is an enlarged SEM image of a second surface 16 of Nonwoven 1.

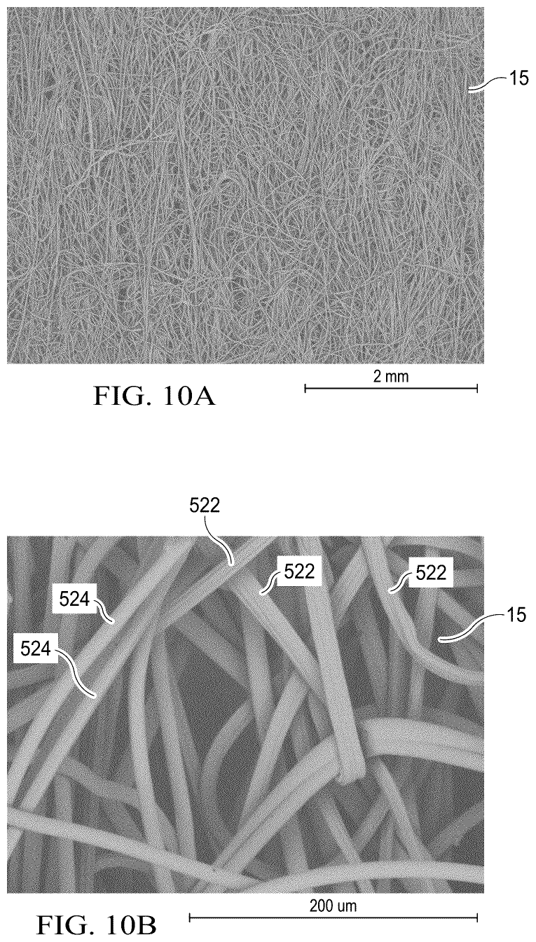

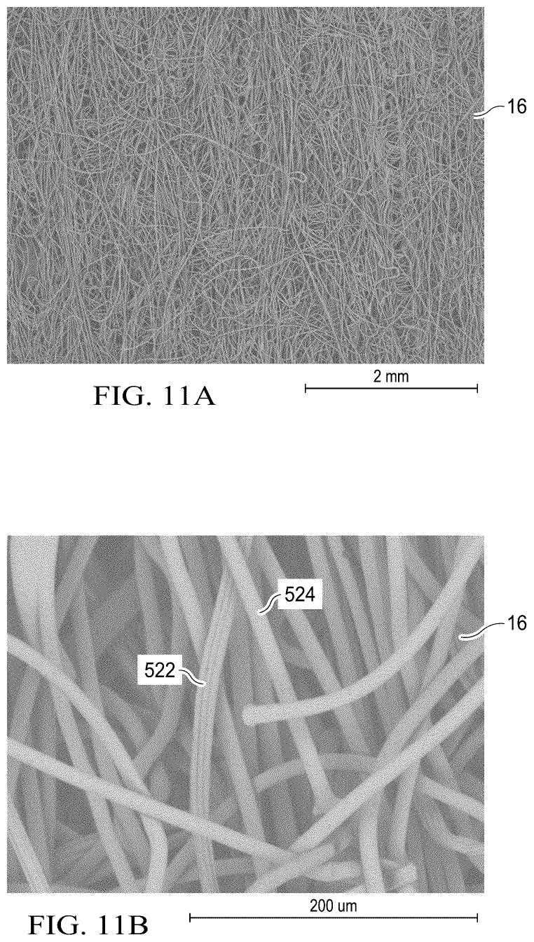

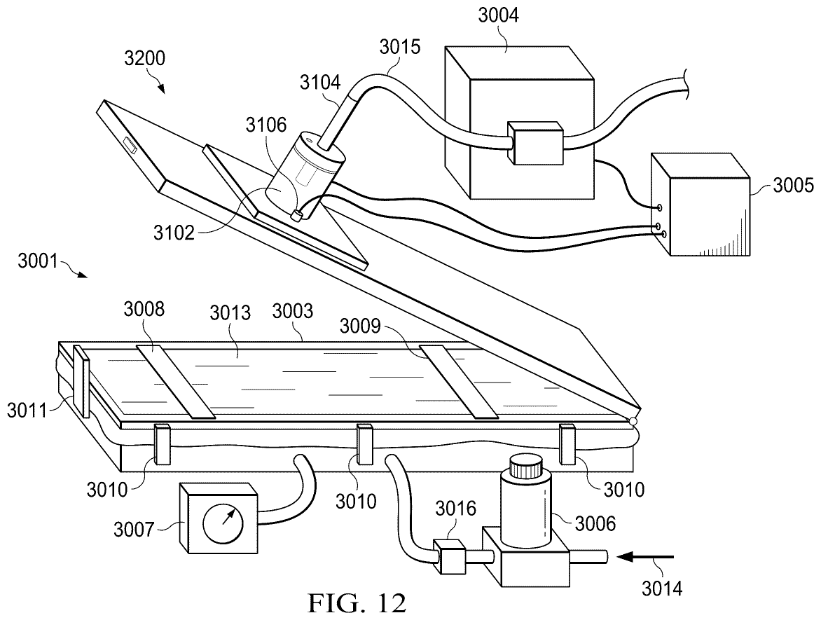

FIGS. 9-11B are SEM images of Nonwoven 2. Referring to FIGS. 9 and 11A, Nonwoven 2 has no apertures. Nonwoven 2 comprises a first stratum and second stratum, the first stratum forming the first surface 15 of Nonwoven 2, and the second stratum forming the second surface 16 of Nonwoven 2. Each of the first stratum and the second stratum comprises absorbing fibers, 1.7 dtex viscose in this case, and synthetic fibers, 1.56 dtex PET fibers 2.2 dtex CoPET/PET fibers in this case. The absorbing fibers and the synthetic fibers are differentiated based on morphology difference. Viscose fibers have a surface with grooves along a fiber length direction while PET fibers and CoPET/PET fibers have a smooth surface. FIG. 10B is an enlarged SEM image of a first surface 15 of Nonwoven 2 which shows the absorbing fibers and synthetic fibers more clearly. FIG. 11B is an enlarged SEM image of a second surface 16 of Nonwoven 2.

Nonwoven 3 has the same nonwoven formulation and structure including a plurality of apertures as Nonwoven 1.

Nonwoven 4 has the same nonwoven formulation as Nonwoven 3, but differs from Nonwoven 3 in such a way that Nonwoven 4 does not have apertures. Nonwoven 5 does not have apertures, either.

Numbers of absorbing fibers and numbers of total fibers of the first stratum and the second stratum in Nonwovens 1 and 2 were measured according to Counting of Fiber Test disclosed herein, and results are indicated in Table 2 below.

Aperture dimension and density of Nonwoven 1 were measured according to Aperture Dimension Test disclosed herein. Nonwoven 1 has about 40 apertures/cm², and an aperture length of about 850 mm, and an aperture width of about 300 mm.

Properties of Nonwovens produced were measured. Wicking effect, material softness and air permeability of the nonwovens were tested according to Vertical Wicking Height Test, Stiffness Test, Air Permeability Test disclosed herein, respectively. All results are indicated in Table 2 below.

TABLE 2

| | | Nonwoven 1 | Nonwoven 2 | Nonwoven 3 | Nonwoven 4 | Nonwoven 5 |
|---|---|---|---|---|---|---|
| Nonwoven base weight | | 43 | 50 | 40 | 40 | 40 |
| (gsm) | | | | | | |
| 2^{^{nd}} | Basis weight | 21.5 | 25 | 20 | 20 | 20 |
| stratum | (gsm) | | | | | |
| | 1.7dtex | 60 | 35 | 60 | 60 | 35 |
| | viscose (wt %) | | | | | |
| | 1.56dtex PET | 40 | 50 | 40 | 40 | 50 |
| | (wt %) | | | | | |
| | 2.2 dtex | | 15 | | | 15 |
| | coPET/PET(wt %) | | | | | |
| 1^{st} | Basis weight | 21.5 | 25 | 20 | 20 | 20 |
| stratum | (gsm) | | | | | |
| | 1.7dtex | 10 | 35 | 10 | 10 | 35 |
| | viscose (wt %) | | | | | |
| | 1.56dtex PET | 90 | 50 | 90 | 90 | 50 |
| | (wt %) | | | | | |
| | 2.2 dtex | | 15 | | | 15 |
| | coPET/PET | | | | | |
| | (wt %) | | | | | |

| | | | |
|---|---|---|---|
| Number of first | 38 | 50 | |
| absorbing fibers in the | | | |
| first stratum | | | |
| Total fiber numbers in | 179 | 183 | |
| the first stratum | | | |
| First absorbing fibers in | 21 | 27.3 | |
| the first stratum (%) | | | |
| Number of second | 102 | 64 | |
| absorbing fibers in the | | | |
| second stratum | | | |
| Total fiber numbers in | 190 | 174 | |
| the second stratum | | | |
| Second absorber fibers in | 53.7 | 36.7 | |
| the second stratum (%) | | | |
| Vertical wicking height | 55 | 40 | |
| (mm) | | | |
| MD stiffness (gf) | 4 | 12 | |
| CD stiffness (gf) | 23 | 42 | |
| Dry air permeability | 199.4 | 124.4 | 151.4 |
| (m³/m²/min) | | | |
| Wet air permeability | 42.34 | 0.37 | 1.77 |
| (m³/m²/min) | | | |
| Ratio of wet air | 21 | 0.3 | |
| permeability to dry air | | | |
| permeability (%) | | | |

Nonwoven 1 of the present invention exhibits a significantly high vertical wicking height compared to Nonwoven 2, a comparative nonwoven, despite a lower basis weight.

Further, Nonwoven 1 exhibits a significantly lower MD stiffness and CD stiffness compared to Nonwoven 2.

Nonwoven 1 of the present invention exhibits a higher air permeability in wet condition than Nonwoven 2 in spite of a high amount of absorbing fibers in the second stratum, i.e., the bottom stratum.

### Example 2. Diaper Preparation

ADS 1-5 were prepared according to Table 3 below, and Diapers 1-5 were fabricated according to Table 4.

TABLE 3

| | ADS 1 | ADS 2 | ADS 3 | ADS 4 | ADS 5 |
|---|---|---|---|---|---|
| Total | 73 | 80 | 70 | 70 | 70 |
| basis | | | | | |
| weight | | | | | |
| (gsm) | | | | | |
| 1^{st} layer | Nonwoven | Nonwoven | Nonwoven | Nonwoven | Nonwoven |
| | 1 | 2 | 3 | 4 | 5 |

| | |
|---|---|
| 2^{nd} layer | 30 gsm, 2D/4D PE/PET carded air-through nonwoven |

TABLE 4

| Diaper No. | |
|---|---|
| 1 | Same as Pampers Hajimeteno Haden Ichiban, size S |
| | (P&G Japan Godo Kaisha, Japan) except replacing |
| | an acquisition-distribution system between a topsheet |
| | and an absorbent core with ADS 1* |
| 2 | Pampers Hajimeteno Hadaeno Ichiban, size S |
| | (P&G Japan Godo Kaisha, Japan) which contains ADS 2* |
| 3 | Same as Pampers Hajimeteno Hadaeno Ichiban, size S |
| | (P&G Japan Godo Kaisha, Japan) except replacing an |
| | acquisition-distribution system between a topsheet |
| | and an absorbent core with ADS 3* |
| 4 | Same As Pampers Hajimeteno Hadaeno Ichiban, size S |
| | (P&G Japan Godo Kaisha, Japan) except replacing an |
| | acquisition-distribution system between a topsheet |
| | and an absorbent core with ADS 4* |
| 5 | Same as Pampers Hajimeteno Hadaeno Ichiban, size S |
| | (Procter and Gamble Japan K.K., Japan) except |
| | replacing an acquisition-distribution system between a |
| | topsheet and an absorbent core with ADS 5* |

*ADS has a width of 90 mm and the same length as an absorbent core.

### Example 3. Diaper Properties

Acquisition time and rewet of Diapers 1 and 2 were tested according to the Modified Fluid Acquisition Test and Light Touch Dryness Test disclosed herein, respectively, and results are indicated in Table 5. Run-off of Diapers 3-5 were tested according to the Run-off Test disclosed herein, and results are indicated in Table 5.

TABLE 5

| | | Diaper 1 | Diaper 2 | Diaper 3 | Diaper 4 | Diaper 5 |
|---|---|---|---|---|---|---|
| Acquisition time | First gush | 67.6 | 74.6 | | | |
| | (75 ml) | | | | | |
| | 2^{nd} gush | 66.9 | 66.3 | | | |
| | (75 ml) | | | | | |
| | 3^{rd} gush | 100.8 | 103 | | | |
| | (75 ml) | | | | | |
| | 4^{th} gush | 221 | 227.5 | | | |
| | (75 ml) | | | | | |
| Rewet (g) | | 0.11 | 0.21 | | | |
| Run-off (%) | | | | 1.06 | 6.97 | 15.82 |

Diaper 1 according to the present invention exhibits a significant lower acquisition time at a first gush, and a significantly lower rewet value than Diaper 2 which is a comparison diaper. Diaper 3 according to the present invention exhibits a far smaller run-off value than Diapers 4 and 5, comparison diapers.

The dimensions and values disclosed herein are not to be understood as being strictly limited to the exact numerical values recited. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising a topsheet, a backsheet, an absorbent core disposed between the topsheet and the backsheet, and an acquisition-distribution system (“ADS”) disposed between the topsheet and the absorbent core, the ADS comprising a first layer comprising nonwoven,
wherein the first layer comprises a first stratum facing towards the topsheet and comprising first absorbing fibers, a second stratum facing towards the backsheet and comprising second absorbing fibers, and a plurality of apertures defined in the first layer; and
wherein a percentage of the second absorbing fibers in the second stratum is at least about 15% higher than a percentage of the first absorbing fibers in the first stratum, as measured according to the Fiber Number Test.

2. The absorbent article according to claim 1, wherein the first layer has a vertical wicking height no lower than about 40 mm, as measured according to Vertical Wicking Height Test.

3. The absorbent article according to claim 1, wherein the first layer has a wet air permeability no lower than about 15 m³/m²/min, as measured according to Air Permeability Test.

4. The absorbent article according to claim 1, wherein the first layer has a ratio of a wet air permeability to a dry air permeability no lower than about 10%, when the wet air permeability and the dry air permeability are measured according to Air Permeability Test.

5. The absorbent article according to claim 1, wherein the apertures have an aperture length in the range from about 100 μm to about 5000 μm.

6. The absorbent article according to claim 1, wherein the first layer comprises apertures in the range of about 10 numbers/cm² to about 200 numbers/cm².

7. The absorbent article according to claim 1, wherein the first absorbing fibers and the second absorbing fibers are the same fibers.

8. The absorbent article according to claim 1, wherein the first absorbing fibers and the second absorbing fibers are the different fibers.

9. The absorbent article according to claim 1, wherein at least one of the first stratum and the second stratum comprises synthetic fibers.

10. The absorbent article according to claim 9, wherein the synthetic fibers have a linear density in the range from about 0.3 dtex to about 15 dtex.

11. The absorbent article according to claim 1, wherein the ADS comprises a second layer comprising thermoplastic fibers in such a way the second layer is positioned between the topsheet and the first layer.

12. A nonwoven comprising a first layer that comprises a first stratum and a second stratum, the first stratum comprising first absorbing fibers and synthetic fibers, the second stratum comprising second absorbing fibers and synthetic fibers, and a plurality of apertures, wherein a percentage of the second absorbing fibers in the second stratum is at least about 15% higher than, a percentage of the first absorbing fibers in the first stratum, as measured according to the Fiber Number Test.

13. The nonwoven according to claim 12, wherein the first layer has a vertical wicking height no lower than about 40 mm, as measured according to Vertical Wicking Height Test.

14. The nonwoven according to claim 12, wherein the first layer has a wet air permeability no lower than about 15 m³/m²/min, as measured according to Air Permeability Test.

15. The nonwoven according to claim 12, wherein the first layer has a ratio of a wet air permeability to a dry air permeability no lower than about 10%, when the wet air permeability and the dry air permeability are measured according to Air Permeability Test.

16. The nonwoven according to claim 12, wherein nonwoven comprises a second layer comprising thermoplastic fibers in such a way that the second layer is positioned above the first layer.

17. The nonwoven according to claim 12, wherein the first absorbent fibers and the second absorbing fibers are the same.

18. An absorbent article comprising a topsheet, a backsheet, an absorbent core disposed between the topsheet and the backsheet, and an acquisition-distribution system (“ADS”) disposed between the topsheet and the absorbent core, the ADS comprising a first layer comprising nonwoven,
wherein the first layer comprises a first stratum facing towards the topsheet and comprising first absorbing fibers, a second stratum facing towards the backsheet and comprising second absorbing fibers, and a plurality of apertures defined in the first layer;
wherein a percentage of the second absorbing fibers in the second stratum is at least about 15% higher than a percentage of the first absorbing fibers in the first stratum, as measured according to the Fiber Number Test;
wherein the apertures have an aperture length in the range from about 100 μm to about 5000 μm; and
wherein the first layer comprises apertures in the range of about 10 numbers/cm² to about 200 numbers/cm².

19. The absorbent article according to claim 18, wherein the first absorbing fibers and the second absorbing fibers are the same fibers.

20. The absorbent article according to claim 18, wherein the first absorbing fibers and the second absorbing fibers are the different fibers.
